(19) United States
(12) Patent Application Publication
(10) Pub. No.: US 2026/0355487 A1
(43) Pub. Date: Nov. 12, 2026

(54) DISTRIBUTION MODULE FOR A DEVICE INTENDED FOR APPLYING A COLORING AGENT AND DEVICE USING SUCH A MODULE

(71) Applicant: ARMINK TATTOO BODY ART, Jougne (FR)

(72) Inventors: Steven KAUFFMANN, Jougne (FR); Eric MARGUET, Les Fins (FR)

(73) Assignee: ARMINK TATTOO BODY ART, Jougne (FR)

(21) Appl. No.: 19/134,931

(22) PCT Filed: Dec. 4, 2023
(86) PCT No.: PCT/IB2023/062178

§ 371 Date: Jun. 2, 2025

(30) Foreign Application Priority Data
Dec. 6, 2022 (FR) FR2212802

(51) Int. Cl. A61M 37/00

(52) U.S. Cl. CPC A61M 37/0084

(57) ABSTRACT

The present invention relates to a distribution module for a device for applying a coloring agent for permanent makeup or tattooing, said distribution module being characterized in that it comprises: a channel, at least one needle arranged in said channel, a plunger, a product reservoir, said reservoir comprising a lumen opening into said channel, and an automatic valve.

### FIELD OF THE INVENTION

The present invention relates to the field of permanent makeup or tattooing.

More specifically, this invention relates to a distribution module for a device intended for applying a coloring agent, this distribution module comprising a channel and at least one needle movable longitudinally in said channel, between a retracted position, in which a distal end of the needle is located inside the channel, and an extended position, in which the distal end of the needle protrudes from the channel. The invention also relates to a device for applying a product, comprising such a module.

### PRIOR ART

Currently, the devices intended for applying a coloring agent to a person and used for permanent makeup or tattooing are formed of a gun in which a tattoo needle moves longitudinally, and of a motor intended to move this needle. When a coloring agent such as in particular an ink is to be applied on or under a user's skin, the needle is dipped in an ink reservoir, then it is applied to the user's skin so as to make the ink penetrate under the user's skin. When the ink which has been taken up by the needle is consumed, this needle is again dipped in the ink reservoir, then the cycle begins again.

A problem that can occur with this type of devices is that the amount of ink that can be used between each dip in the reservoir is relatively small because it is a capillary reservoir. Thus, when the person who performs the tattooing has to draw, for example, a straight or elongated line, they must regularly stop the drawing to take more ink. Resuming the drawing at the exact point where it was stopped is relatively difficult and it is possible that the drawing will not be ideal for this reason.

Furthermore, the time required to perform a tattoo can be long because of the ink refills that need to be done regularly. This is all the more important, especially when large areas need to be colored, for example when shading. This relatively long working time can be unpleasant for the person who performs the tattoo or makeup, but especially for the person on whom the tattoo or makeup is performed. Indeed, marking under the skin can be relatively painful. A shorter working time therefore brings more comfort to the person on whom the marking is performed.

Another problem can arise from the fact that the reservoirs containing the ink are open to allow ink to be drawn. This ink is therefore in contact with the air. It is then possible that it is contaminated by pathogens which can then be injected under the skin of the person on whom the tattoo or makeup is performed, which can cause infections.

To overcome some of these disadvantages, there is a tattoo or permanent makeup device comprising a reservoir integrated into the gun. Such a device is described in U.S. Pat. No. 6,345,553. This document describes a tattoo or permanent makeup gun comprising a module in which a tattoo needle moves longitudinally under the impulse of a motor. The tattoo gun comprises an ink reservoir. The needle, during its alternating longitudinal displacement, moves in the ink reservoir, then its free end protrudes from the tattoo gun so as to be able to apply the ink taken by the needle, under the user's skin.

This embodiment largely avoids refilling ink in an external reservoir. It is therefore possible to draw relatively long lines and fill large areas without having to refill ink. In addition, the ink is not exposed to the air and the risks of contamination or infections are therefore minimized.

This gun, however, has other disadvantages. Indeed, the presence of a plunger, namely the part supporting the needle and also the flexible sealing parts, during the tattooing action, creates overpressure pulses causing discharges expelling the ink in an uncontrollable manner.

In addition, the ink is constantly in contact with the metal needle and chemical integrations can occur. In the case of storage for several weeks, contamination of the ink becomes problematic and the needle can become blunt due to slow micro-corrosion. In addition, it should be noted that the ink is composed of solvents as well as more or less solid microparticles. The presence of the needle in the ink with even a slumen loss of solvent causes accumulations, or films, on the surface of the needle, degrading its effectiveness. Another limitation comes from the shape of the needles. Indeed, to create specific tattoos, the needle is composed of several points welded together. This assembly of points imposes non-circular shapes as soon as the needle is composed of more than one point. It is then very difficult, even impossible, to create a seal in the front spout, making the reservoir sensitive to contamination and leaks without the use of an additional sealing cap which must be replaced after each use.

Another disadvantage is that ink is introduced into the reservoir, and then the needle moves within this reservoir. It is not possible to easily empty the ink from the reservoir and replace it with ink of a different color. Indeed, this would result in a heterogeneous mixture of colors that would not be usable. Thus, if the ink contained in the reservoir is not completely consumed, it is lost.

Furthermore, the ink reservoir in which the needle moves is located in a position relatively far from the free end of the needle, that is to say from the end through which the needle opens to carry out the tattoo. To have ink take up by the needle, its free end must enter entirely into the reservoir. This requires the needle to be guided in a particularly rigid manner, which complicates the production of the gun from a mechanical point of view and which makes it more expensive. In addition, due to the construction of the gun, it is necessary for ink to be available on the side of the reservoir where the needle opens. This requires the person who performs the tattoo to hold the gun in a certain way, with the needle substantially vertical and pointing downwards, otherwise the needle does not receive ink. This makes this gun impractical to use. As a result, the solution is not ideal, particularly because the ink does not easily come into contact with the needle. Furthermore, the device described in this document does not allow the use of a removable reservoir.

Another disadvantage of this device comes from the fact that the needle is constantly immersed in ink. This means that the only ink that can be used is that contained in the reservoir. Therefore, it is not possible to use the ink of the reservoir for one part of a tattoo, then use the device by dipping the needle in the ink of an external reservoir to carry out another part of the tattoo.

As a result, the solution described in U.S. Pat. No. 6,345,553 is not ideal in practice. Indeed, it is not possible to change ink color while keeping the same gun. The unused ink is lost and the gun must be handled in a certain way, otherwise it does not work. In addition, it is not possible to use the device alternately in a “conventional” mode in which the needle is dipped in an external ink reservoir, and in the mode using the ink contained in the reservoir integrated into the device.

To solve the technical problems listed above, document WO 2021/204971 describes a distribution module for a device intended for the application of a coloring agent for permanent makeup or tattooing. This distribution module comprises a channel and at least one needle movable longitudinally in the channel, between a retracted position, in which a distal end of the needle is located inside the channel, and an extended position, in which the distal end of the needle protrudes from the channel. The distribution module further comprises a reservoir of coloring agent, which communicates with the channel only in an area located downstream of the needle when this needle is in a certain position

This distribution module does not always allow the flow rate of coloring agent delivered by the reservoir to be satisfactorily adjusted. This can lead to irregularities in the tattoo, as well as leaks of product at the front and/or rear of the distribution module. These inconveniences are caused by the rapid back-and-forth movements of the plunger.

There is therefore a need for a device intended for the application of a coloring agent, via at least one needle, which is flexible and practical to use, that is to say in particular which allows continuous use without having to dip the needle in product regularly, which allows a change of product without causing waste, which can be used in the normal positions of use of such a device, and which allows the flow rate of product to be controlled. All this while having a sealed reservoir and without prolonged contact between the needle and the coloring agent.

### Disclosure of the Invention

These objectives, as well as others which will appear more clearly hereinafter, are achieved using a distribution module for a device intended for applying a coloring agent for permanent makeup or tattooing, said distribution module being characterized in that it comprises:
a channel,
at least one needle, arranged in said channel,
a plunger, arranged in said channel, upstream of said needle, said needle being secured in translation to said plunger, the assembly formed by said needle and said plunger being movable longitudinally in said channel between a retracted position, in which a distal end of the needle is located inside said channel, and an extended position, in which the distal end of the needle protrudes from said channel,
a product reservoir, said reservoir comprising a lumen opening into said channel, and
an automatic valve, said automatic valve being formed by the cooperation between at least one slit, arranged on the periphery of said plunger, and said lumen, said at least one slit being arranged so as to be located in line with said lumen when said plunger is in a first intermediate product supply area, located between the retracted position and the extended position, the lumen being closed by said plunger outside said first intermediate product supply area.

The distribution module may comprise a front chamber, arranged in said channel, downstream of said lumen, said slit being able to be located in said front chamber when said plunger is located in a second intermediate product supply area.

The distribution module may comprise one or several decompression chimney(s), opening into said front chamber.

The decompression chimney may comprise a set of grooves, to allow, by capillarity, the rise of product in the decompression chimney.

The decompression chimney can be integrated into said reservoir.

The cooperation of the slit and the lumen can further allow air to return to the reservoir.

The lumen may be shut off by a plug before using said reservoir.

According to one embodiment of the invention, the plunger comprises at least one slit having a constant shape extending over the entire circumference of the plunger.

According to another embodiment of the invention, said plunger has a cylindrical shape, said plunger comprising several slits of different dimensions, arranged at different positions on the circumference of the plunger, the plunger being movable in rotation about its axis, to allow the selection of the slit to be used for the supply of product.

According to another embodiment of the invention, the distribution module comprises several needles arranged in the same plane, said slit having a substantially rectangular shape and being arranged on said plunger so as to be located in line with said lumen when said plunger is located in said first intermediate product supply area.

The reservoir may comprise a bottom, the reservoir and/or said bottom being inclined relative to the axis of said channel.

The angle of inclination between the bottom of said reservoir and the axis of said channel is for example comprised between 5° and 55°.

The bottom of the reservoir may comprise a set of grooves, to allow, by capillarity, the rise of product in the bottom of said reservoir.

The reservoir may be removable.

The removable reservoir may comprise a member for connecting said removable reservoir to said distribution module, said connecting member comprising an element for shutting off said lumen, movable between a closed position, in which the shutter element shuts off said lumen, and an open position, in which the shutter element releases said lumen, the shutter element being moved from said closed position to said open position when said removable reservoir is mounted on said distribution module.

The removable reservoir may comprise means for distributing the product in said reservoir.

The reservoir may comprise a plug, removably mounted on a filling opening of said reservoir.

The plug may be a bayonet plug.

The reservoir may comprise a cover, making it possible to close a filling opening of said reservoir.

The reservoir may comprise an air inlet hole.

Said shutter element may be arranged to shut off the air inlet hole when it is in said closed position, said shutter element being arranged to release the air inlet hole when it is in said open position.

The needle and the plunger can be made integral by inserting a proximal end of said needle into said plunger, along its longitudinal axis.

The front part of said channel may have the shape of an elongated spout.

The invention also relates to a device for applying a coloring agent for permanent makeup or tattooing, this device being characterized in that it comprises a distribution module as described above, and a module for actuating the needle of the distribution module.

### LIST OF FIGURES

The invention will be better understood on reading the following description of preferred embodiments, given as a simple figurative and non-limiting example, and accompanied by the figures among which:

FIG. 1 is a longitudinal sectional view of a distribution module of a device for applying a product, according to a first embodiment of the invention;

FIG. 2 is a view similar to FIG. 1, showing a plunger of the distribution module in a first intermediate displacement position;

FIG. 3 is a view similar to FIG. 1, showing a plunger of the distribution module in a second intermediate displacement position;

FIG. 4 is a view similar to FIG. 1, showing the plunger in an extended position;

FIG. 5 is a top view of a distribution module, according to a second embodiment of the invention;

FIG. 6 is a perspective view of the distribution module of FIG. 5, a cover being arranged on a reservoir of the distribution module;

FIG. 7 is a longitudinal sectional view of a distribution module, according to a third embodiment of the invention;

FIG. 8 is a longitudinal sectional view of a distribution module, according to a fourth embodiment of the invention;

FIG. 9 is a perspective view of a distribution module, according to a fifth embodiment of the invention, in which the distribution module comprises a removable reservoir;

FIG. 10 is a longitudinal sectional view of the distribution module of FIG. 9;

FIG. 11 is a view similar to FIG. 9, showing the removable reservoir mounted on the distribution module;

FIG. 12 is a longitudinal sectional view of the distribution module of FIG. 11;

FIG. 13 is a longitudinal sectional view of a distribution module, according to a sixth embodiment of the invention, and

FIG. 14 is a perspective view of a distribution module according to a seventh embodiment of the invention.

### DETAILED DESCRIPTION OF EMBODIMENTS OF THE INVENTION

The invention relates to a device for applying a coloring agent for permanent makeup or tattooing.

According to the present invention, the term “coloring agent” means any colored, natural or synthetic, substance which, when brought into contact with a support, under appropriate conditions, is fixed to the latter in a lasting manner, imparting a certain color to it. The terms “coloring agent”, “ink”, “colorant”, “color”, “dye” will be used in the present description in an indifferent and substitutable manner.

The coloring agent is for example ink.

Usable inks are for example Panthera Ink (registered trademark) or Eclipse Black Tattoo Ink (registered trademark).

The device is essentially formed of two parts, namely a distribution module 1, comprising in particular a needle 2, and a module for actuating the needle 2. This needle 2, or a support for the latter, allows, during a movement linked to its use, the coloring agent to be released or not.

According to a preferred embodiment, the distribution module 1 is removable, so that a distribution module 1 can be mounted on the actuation module, be detached therefrom and be replaced by another distribution module 1.

In practice, the actuation module comprises at least one motor and is used to carry out a large number of tattoos while several distribution modules 1 can generally be used for one person. For hygiene reasons, a distribution module is generally single-use, that is to say it is used to tattoo a single person and is disposed of after use on that person.

The actuation module may be of any conventional type and is therefore neither described nor represented in detail in the figures. It is simply designed to receive, in a removable manner, a distribution module 1.

### 1. Distribution Module

The distribution module 1 comprises a body 3, to allow it to be placed in the actuation module.

The length of the module 1 is for example comprised between three and twenty centimeters.

The distribution module 1 also comprises at least one needle 2, a plunger 4, and a product reservoir 5.

Apart from the needle 2, all of the parts in contact with the ink are made from a material that is inert vis-à-vis this ink.

In the case of the use of a distribution module supplied with pre-filled ink, the plunger 4 can be equipped with a blocking of its longitudinal movement by, for example, a rotation of the latter. This system makes it possible to secure the distribution of ink, typically during transport.

The reservoir 5 can be filled almost completely beforehand in order to limit the presence of air, with the aim of limiting the risks of overpressure, and therefore of leaks, during airplane journeys or increase in the storage temperature, for example.

The distribution module 1 further comprises a longitudinal channel 6, in which the needle 2 undergoes an alternating back-and-forth movement, by means of the plunger 4, under the effect of the actuation module.

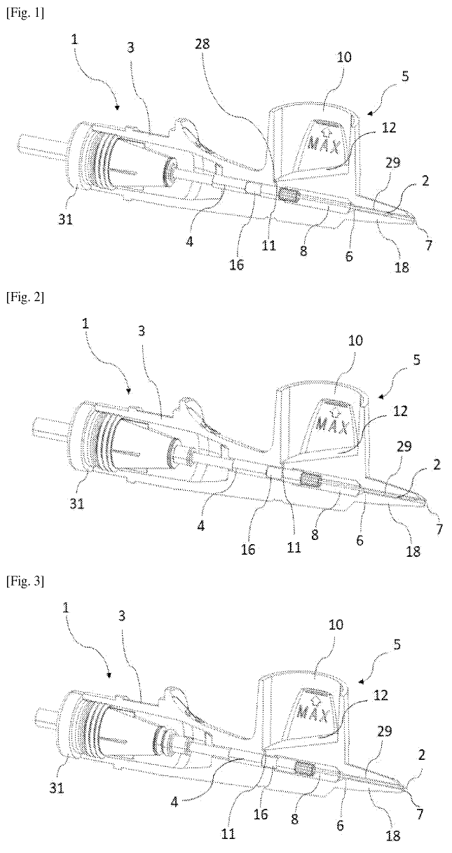

The needle 2 and the plunger 4 are secured in translation. The assembly formed by the needle 2 and the plunger 4 is movable longitudinally in the channel 6 between a retracted position (FIG. 1), in which a distal end of the needle 2 is located inside the channel 6, and an extended position (FIG. 4), in which the distal end of the needle 2 protrudes from the channel 6.

The channel 6 opens at one end 7 of the distribution module 1, called the free end, through which the needle 2 can protrude from the channel 6 and come into contact with the skin of the person to be tattooed so as to deposit product there.

The channel 6 comprises a front chamber 8, capable of containing product. The term “front chamber” refers to the downstream part of the channel 6, in which the distal end of the needle 2 is located when it is in the retracted position. The front chamber 8 is arranged close to the free end 7. The needle 2 can thus recover a small quantity of product on each return trip between the retracted position and the extended position.

The reservoir 5 may be fixed and form an integral part of the distribution module 1, or be removable. In this second case, we refer below to a removable cartridge 105.

The distribution module 1 may furthermore advantageously comprise a decompression chimney 9.

### 2. Reservoir

The reservoir 5 comprises a sealed wall and a filling opening 10.

The reservoir 5 further comprises a lumen 11 opening into the channel 6, to allow the passage of product from the reservoir 5 to the channel 6.

Before using the reservoir 5, the lumen 11 can be shut off by a plug 28 (FIG. 1). The plug 28 can be made from a fluid that is not soluble in the product, for example a fatty fluid released by a certain movement, or in a solid material released by a certain movement. The plug 28 is arranged to be broken by the displacement of the plunger 4, at the start of use. The plug 28 makes it possible to avoid leakage in the case of an extremely capillary product via the lumen 11 before the reservoir 5 is used.

When the lumen 11 is arranged on the side of the reservoir 5 opposite the free end 7, the reservoir 5 advantageously comprises a bottom 12 inclined relative to the axis of the channel 6. The angle of inclination between the bottom 12 of the reservoir 5 and the axis of the channel 6 is for example comprised between 5° and 55°, preferably between 15° and 35°. This feature has the function of promoting the presence of product at the lumen 11, even when the module 1 is inclined, which is often the case during its use. Alternatively, the reservoir 5 could itself be inclined relative to the axis of the channel 6.

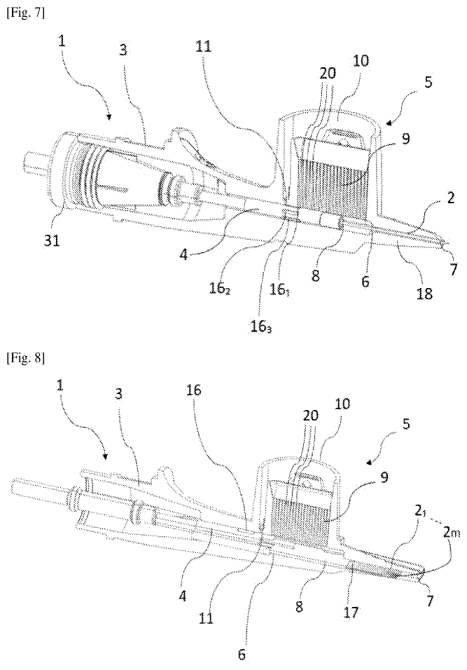

The bottom 12 of the reservoir 5 may further comprise a set of grooves 13 (visible in FIG. 5), to allow, by capillarity, the rise of product in the bottom 12 of the reservoir 5, towards the lumen 11. This feature makes it possible to further promote the presence of product at the lumen 11, in particular when the module 1 is inclined. The lumen 11 participates in the management of the product flow rate.

The reservoir 5 may comprise a plug, removably mounted on the filling opening 10 of the reservoir 5. The plug is for example a bayonet plug.

The reservoir 5 may comprise, in addition to or instead of the plug, a cover 14 (visible in FIG. 6), to hermetically close the filling opening 10 before its mounting. The cover 14 may comprise, in a conventional manner, a tab 15 to allow it to be easily removed.

When the reservoir 5 is intended to be used while remaining hermetically closed by a plug or a cover 14, it further comprises an air inlet hole, to allow atmospheric pressure to be maintained inside the reservoir 5, even when the quantity of product inside the reservoir 5 is modified. The air inlet hole may for example be provided at the plug, or be obtained by piercing the cover 14 just before using the reservoir 5. Depending on the fluidity of the ink, the cover 14 may or may not cover the chimney 9.

In the case of using fluid ink, the entire reservoir 5 can remain constantly completely sealed. In this case, the pressures are balanced by the rise of air bubbles coming from the chimney 9 and passing into the slit 16 when the plunger is activated.

### 3. Needle and Plunger

The needle 2 is arranged in the channel 6. The plunger 4 is also arranged in the channel 6, upstream of the needle 2. The plunger 4 and the needle 2 are secured in translation. For this, for example, a proximal end of the needle 2 is fixed inside the plunger 4.

The plunger 4 is arranged so as to be able to be driven in translation by the actuation module, when the distribution module 1 is mounted on the actuation module.

The plunger 4 further serves as a guide member for the needle 2. This has the advantage that the needle 2 is always guided adequately.

The plunger 4 comprises at least one slit 16 arranged on its periphery. The function of the slit 16 is to allow the supply of product from the reservoir 5 to the front chamber 8.

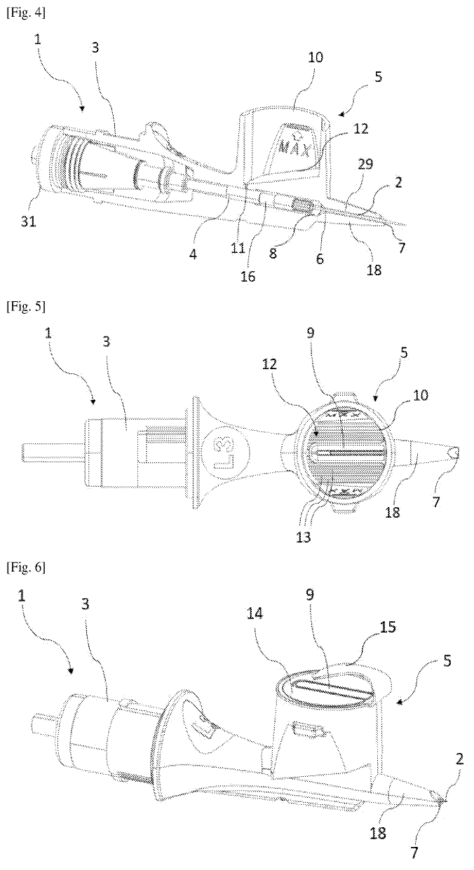

The slit 16 is arranged so as to be located in line with the lumen 11 of the reservoir 5, when the plunger 4 is in a first intermediate product supply area (FIG. 2 and FIG. 3). The first intermediate area is located between the retracted position and the extended position. When the slit 16 is located in line with the lumen 11, product is transferred into the slit 16 from the reservoir 5, via the lumen 11.

Outside the first intermediate supply area, the plunger 4 shuts off the lumen 11. The plunger 4 shuts off the lumen 11 in particular in the retracted position. This feature makes it possible to avoid product leaks when the module 1 is not in use.

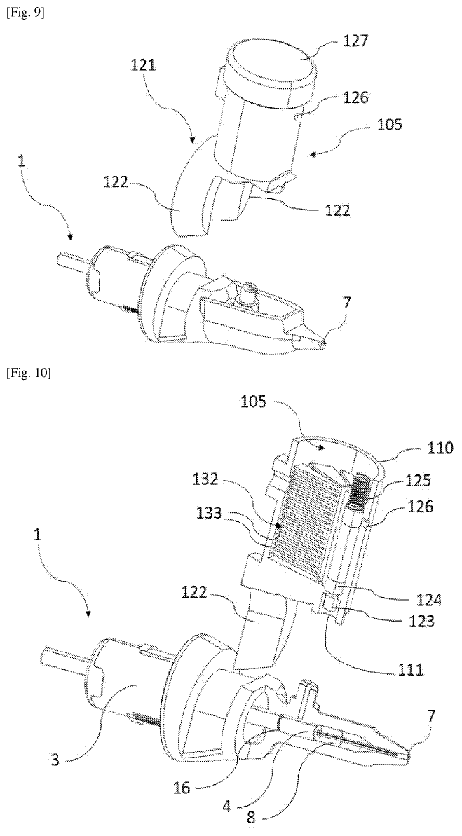

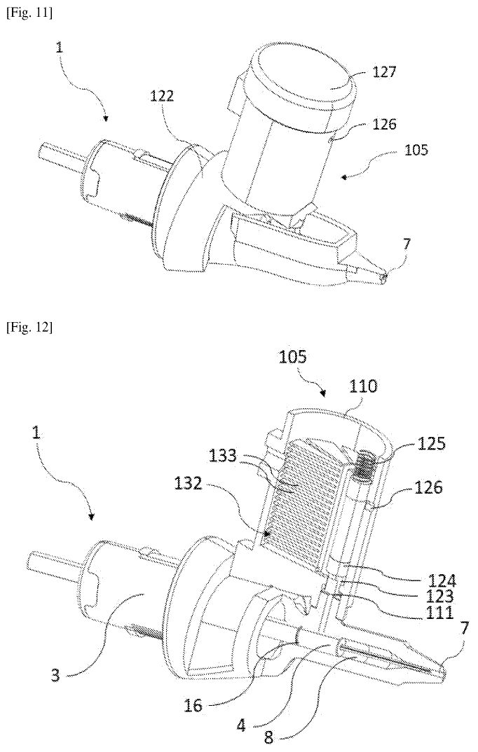

According to embodiments of the invention, represented for example in FIGS. 1 to 4 and 9 to 13, the plunger 4 has a cylindrical shape, and comprises a slit 16 of cylindrical shape extending over a complete circumference of the plunger 4. The length of the slit 16 along the longitudinal axis of the plunger is selected according to the desired flow rate of product. Indeed, the longer the slit 16, the greater the quantity of product that will pass from the reservoir 5 to the slit 16 during the stroke of the plunger 4. This feature therefore makes it possible to adjust the flow rate of product. This feature also makes it possible, when setting the output of the needle 2, to automatically increase the quantity of ink.

According to another embodiment of the invention, represented in FIG. 7, the plunger 4 has a cylindrical shape, and comprises several slits 161 to 16n, n being a positive integer. The slits 161 to 16n each have a substantially rectangular shape and are of different dimensions. The slits 161 to 16n are arranged at different positions on a circumference of the plunger 4. The number of slits 161 to 16n is for example four. This number is not limiting.

When the plunger 4 comprises several slits 161 to 16n, the plunger 4 is movable in rotation about its axis, to allow the selection of the slit 161 to 16n to be used for supplying the product. The rotation of the plunger 4 can be carried out manually. The slit 161 to 16n which will be located in line with the lumen 11 during the stroke of the plunger 4 is called the «slit to be used». This feature makes it possible to adjust the flow rate of product even more precisely, and to modify this flow rate during the tattooing process.

According to another embodiment of the invention, shown in FIG. 8, the module 1 comprises several needles 21 to 2m, m being a positive integer, arranged in the same plane. The plunger 4 here comprises a slit 16 of substantially rectangular shape, arranged so as to be located in line the lumen 11 in the first intermediate supply zone. As described above, the dimensions of the slit 16 are selected according to the desired product flow rate.

The needle 2 can be fixed to the plunger 4 by inserting the proximal end of the needle 2 into the plunger 4, along its longitudinal axis. The proximal end can further be bonded to the plunger 4.

Other means can be used to make the needle 2 and the plunger 4 secured in translation. For example, in the case of several needles 21 to 2m arranged in the same plane, a fixing member 17 (FIG. 8) can be provided to receive the proximal end of each needle 21 to 2m. The fixing member 17 is then fixed to the plunger 4.

### 4. Front Chamber

The front chamber 8 of the channel 6 is located downstream of the lumen 11, and extends to the vicinity of the free end 7 of the channel 6.

The term «front chamber» means the downstream part of the channel 6, in which the distal end of the needle 2 is located when it is in the retracted position.

The front chamber 8 extends over a sufficient length of the channel 6 so that the slit 16 of the plunger 4 is located in the front chamber 8 when the plunger 4 is located in a second intermediate product supply zone (FIG. 3 and FIG. 4).

The front chamber 8 is arranged in order to allow the transfer of product from the slit 16 to the front chamber 8, when the plunger 4 is in the second intermediate product supply zone. For this purpose, the front chamber 8 may have a diameter slightly greater than the diameter of the plunger 4, or may comprise longitudinal capillary grooves, for example.

The first and second intermediate product supply zones may be distinct. In this case, the second intermediate product supply zone is located downstream of the first intermediate product supply zone. In other words, in this case, the reservoir 5 supplies the slit 16 with product, then, after displacement of the plunger 4, the slit 16 supplies the front chamber 8 with product.

Alternatively, the first and second intermediate product supply zones may comprise an overlapping zone. In this case, when the plunger 4 is in the overlapping zone, the reservoir 5 supplies the slit 16 with product, and, simultaneously, the slit 16 supplies the front chamber 8 with product.

The body 3 of the module 1 has, at the free end 7, the shape of an elongated spout 18. Here, a spout 18 whose length-to-diameter ratio is greater than four is considered to be «elongated». For example, the spout 18 has a diameter of approximately 1 mm and a length of approximately 4 mm. The use of an elongated spout 18 is made possible by supplying product via a reservoir 5. The advantage of an elongated spout 18 is that it allows better control of the product flow rate and prevents product leaks through the free end 7.

A tube 29 may further be inserted into the channel 6, at the spout 18. The tube 29 may be made of a hydrophilic or hydrophobic material, depending on the applications. The tube 29 may, for example, be made with a coating of Kollidon (registered trademark) or of plasma-treated silicone to manage the hydrophilicity (or hydrophilic character). The function of this tube 29 is to participate in controlling the flow rate of product at the free end 7 and to facilitate the priming during the first use.

All of the parts in contact with the ink may undergo treatments in order to manage the hydrophilicity, always with the aim of facilitating the priming or accelerating it.

### 5. Decompression Chimney

As shown in particular in FIGS. 7, 8 and 14, the module 1 may comprise one or more decompression chimneys 9, located downstream of the lumen 11.

The decompression chimney(s) 9 open into the front chamber 8.

The section of the or each chimney 9 may have a circular, rectangular or ovoid shape, for example.

The decompression chimney 9 is arranged so as to be located in line with the slit 16 when the plunger 4 is in the second intermediate product supply zone.

The decompression chimney 9 makes it possible to manage pressure changes in the front chamber 8 due to the stroke of the plunger 4 and/or the variation in the quantity of product present in the front chamber 8. The utility lies in the elimination of leakage generated by the overpressures of the plunger 4.

The decompression chimney 9 may comprise a set of grooves 20, to allow, by capillarity, the rise of product in the decompression chimney 9. This feature makes it possible to maintain and regulate any excess product in the chimney 9, thus avoiding a leak of product at the free end 7, and to release the product, due to gravity, when necessary. The grooves are, for example, oriented substantially along the longitudinal axis of the decompression chimney 9.

As shown in the figures, the decompression chimney 9 can be integrated into the reservoir 5. In this case, the shape of the cover 14 can be adapted to shut off the filling opening 10 while leaving the upper end of the chimney 9 free, as can be seen in FIG. 6.

The shape of the slit 16, in combination with the lumen 11, must allow the channel 6 to be supplied with ink when the plunger 4 is activated, while allowing air to return to the reservoir 5, in the case where the latter is hermetically closed. This air return, coming from the chimney 9, prevents the reservoir 5 from ceasing to supply the system with ink due to an internal depression in the latter.

### 6. Removable Cartridge

With reference to FIGS. 9 to 12, an embodiment is described below in which the reservoir is removable. Such a reservoir is herein called a removable cartridge 105.

The function of the cartridge 105 is identical to the function of the reservoir 5, namely to allow the supply of product.

The removable cartridge 105 comprises a member 121 for fixing the removable cartridge 105 to the distribution module 1. The fixing member 121 comprises, for example, clipping means 122, intended to be clipped onto the body 3 of the distribution module 1.

The removable cartridge 105 further comprises a member 123 for connecting the cartridge 105 to the channel 6, so as to allow the flow of product from a lumen 111 of the cartridge 105 to the channel 6, when the cartridge 105 is mounted on the distribution module 1.

The connecting member 123 comprises a shutter element 124 of the lumen 111, movable between a closed position, in which it shuts off the lumen 111, and an open position, in which it releases the lumen 111.

The shutter element 124 is displaced from the closed position to the open position when the removable cartridge 105 is mounted on the distribution module 1. This displacement can be carried out automatically at the time of the mounting.

For this purpose, the shutter element 124 may, for example, have the shape of a rod, which is pushed back by a spring 125 towards the closed position. Thus, in the absence of additional force exerted on the spring 125, the shutter element 124 is in the closed position. This position makes it possible, in particular, to prevent product leaks when the cartridge 105 is not in use. Then, when the cartridge 105 is mounted, the cooperation between the rod 124 and the body 3 of the distribution module 1 compresses the spring 125,, thus causing the displacement of the shutter element 124 towards the open position.

The compression of the spring 125 is for example obtained by cooperation between a protrusion arranged on the lower part of the rod 124 and a protrusion of the body 3 of the distribution module 1, this cooperation forcing a displacement of the rod 124 upwards.

The shutter element 124 then remains in the open position as long as the cartridge 105 is mounted on the module 1, because the clipping means 122 hold the entire cartridge 105 in position.

Similar to what has been described for the reservoir 5, the removable cartridge 105 may comprise a plug 127 and/or a cover, to open and close its filling opening 110.

The removable cartridge comprises an air inlet hole 126, to maintain atmospheric pressure inside the removable cartridge 105, even in the presence of a plug 127 or a cover on the filling opening 110.

The shutter element 124 may be arranged to shut off the air inlet hole 126 when it is in the closed position, and to release the air inlet hole 126 when it is in the open position. This feature makes it possible to prevent leaks when the cartridge 105 is not in use.

The shutter element 124 may further comprise means 132 for distributing the product in the cartridge 105, by capillarity. The distribution means 132 comprise fins 133. The distribution means 132 make it possible to improve the regulation of the flow rate.

It should be noted that a removable cartridge 105 may be placed on different distribution modules 1, more precisely distribution modules 1 having needles 2 of different shapes. The same cartridge 105 may, for example, first be placed on a distribution module 1 having a needle 2 specific to the production of fine lines, then the same cartridge 105 may be placed on a distribution module 1 having a needle 2 specific to the production of large surfaces and/or shading.

The use of cartridges 105 also allows the installation of pre-filled cartridges, without it being necessary for these cartridges to be filled by the user himself.

### 7. Operation

The operation of the distribution module 1 is described below.

The distribution module 1 is placed on the actuation module. Then, the motor of the actuation module is started.

When the distribution module 1 is used in a normal operating mode, the assembly formed by the plunger 4 and the needle 2 is displaced longitudinally in the channel 6, under the effect of the actuation module, in a back-and-forth movement, between the retracted position and the extended position.

The use begins with a priming phase, which consists of transferring enough product into the front chamber 8, so that the needle 2 can then take some at each passage, and bring it to the person's skin.

In the retracted position (FIG. 1), the slit 16 is upstream of the lumen 11, 111 of the reservoir 5, 105. When the plunger 4 is displaced to the extended position, the slit 16 enters the first intermediate supply zone (FIG. 2). As a result, the reservoir 5, 105 supplies the slit 16 with product.

Then, when the plunger 4 continues to be displaced towards the extended position, the slit 16 enters the second intermediate supply zone (FIG. 3 and FIG. 4). The product contained in the slit 16 is then released into the front chamber 8.

After one or a few back-and-forth movements, the front chamber 8 contains enough product for the tattooing to begin.

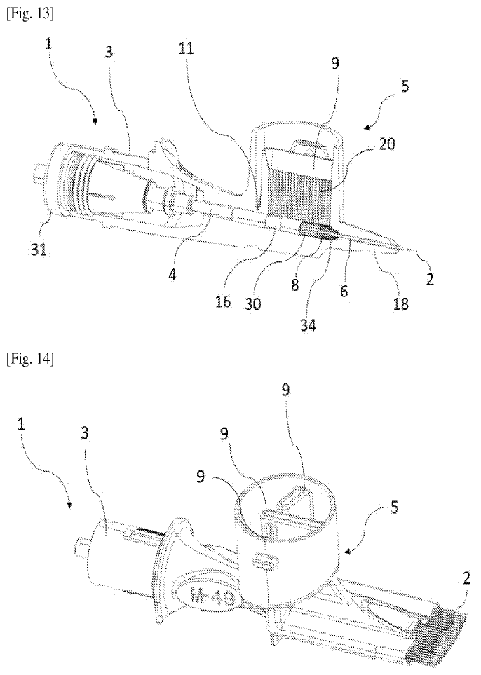

To facilitate the priming of the distribution module 1, a small amount of product may be manually placed in the chimney 9. In combination or instead, grooves 30, 34 (FIG. 13) may be provided on the front portion of the plunger 4 and/or on the inner wall of the front chamber 8, to promote the advancement of the product into the front chamber 8.

At this stage, the assembly formed by the plunger 4 and the needle 2 continues to be displaced longitudinally in the channel 6, under the effect of the actuation module, between the retracted position and the extended position.

At each back-and-forth movement, in parallel with the filling of the front chamber 8 via the slit 16, the needle 2 takes a small quantity of ink, which it can then deposit under the skin of the tattooed person, in a conventional manner.

At each cycle, product is thus brought into the front chamber 8, and the needle 2 recovers it.

The assembly formed by the slit 16 of the plunger 4 and the lumen 11, 111 thus forms an automatic valve. The term «automatic» refers to the fact that the valve is automatically actuated by the actuating module, between a closed position, in which the plunger 4 shuts off the lumen 11, 111 of the reservoir 5, 105, and an open position, in which product can be conveyed to the front of the channel 6 via the slit 16. The automatic valve makes it possible to control and regulate the flow rate of the product.

When the module 1 comprises a decompression chimney 9, this makes it possible to regulate even more precisely the quantity of product recovered by the needle 2. Indeed, any excess product is pushed into the chimney 8, rather than towards the front of the channel 6, which prevents any excess pressure, and therefore any leakage.

When the chimney 9 comprises grooves 20, the excess ink is further retained by capillarity in the chimney 9, until the force of gravity is greater than the capillarity force.

The position of the slit 16 is adapted so that the product cannot flow towards the rear of the module 1 during the return stroke of the plunger 4. A tip 31 may nevertheless be provided to prevent any leakage towards the rear of the module 1, in the case where a small quantity of product is drawn towards the rear of the module 1 via the slit 16.

When the product must be added to the reservoir 5, 105, for example because the contents thereof have been used, the product can simply be added through the opening 10, 110 of the reservoir 5, 105, either by removing the plug 127 and pouring the product into the reservoir 5, 105, or by removing or piercing the cover 14. The quantity of introduced product can be selected so as to correspond substantially to the quantity of required product for the selected product, for example the selected color, which avoids the waste.

In the case of a cartridge 105, this can alternatively be changed, that is to say replaced by a new pre-filled cartridge 105.

According to a variant of the invention, the stroke of the plunger 4 and needle 2 assembly can be adjusted by the actuation module, which makes it possible to manage the quantity of distributed product.

Here, in a first mode of use of the distribution module 1, the stroke is comprised between a first position, in which the slit 16 is upstream of the lumen 11, 111, and a second position, in which the slit 16 is in the front chamber 8. The first mode of use corresponds to the use of the reservoir 5, 105 to convey the product into the channel 6.

In a second mode of use, the stroke is comprised between a first position identical to the first position described above and a second position in which the slit 16 is upstream of the front chamber 8.

In this case, the front chamber 8 is not supplied with the product from the reservoir 5, 105. A product from an external reservoir can then be used.

The present invention makes it possible to use a tattoo or permanent makeup device without having to be regularly stopped to add ink to the tattoo needle 2. It makes it possible to easily and quickly mark large areas and to change colors simply. It can also be used in two different modes, namely a mode in which the ink from the reservoir 5, 105 is used, and another mode in which ink from an external reservoir is used. The mode selection can be done quickly and easily.

This is particularly advantageous in the case where a large part of a tattoo or makeup is done using one color while another part requires the use of another color or in the case where two colors must be used alternately. In such a case, it is not necessary to empty the integrated reservoir 5, 105, clean it and refill it with another color to make a small part of the tattoo or makeup. It is sufficient to close the reservoir 5, 105, that is to say to activate the second mode of use, then to use the device in a conventional manner, by drawing ink with the needle 2 from an external reservoir.

When this part of the tattoo or makeup is finished, the reservoir 5, 105 can be reopened, that is to say to activate the first mode of use, and the ink from the integrated reservoir 5, 105 is used again.

These various features make the device easier and more pleasant to use for the person applying makeup or tattooing. The same operations can be performed in a significantly shorter time than with prior art devices, which is advantageous for the person applying the tattoo, but especially for the person receiving the tattoo. Indeed, since a tattoo can be relatively painful, saving time when applying a tattoo means greater comfort for the person receiving the tattoo.

The distribution module according to the present invention can be used in all sorts of positions limited by the reversal of the reservoir into the chimney, which allows permanent makeup or tattooing to be performed without either the person applying the tattoo or the person receiving it having to be placed in an uncomfortable position. Increasing the height of the reservoir and the chimney allows for an increase in the permissible angles of use.

Furthermore, with the device of the invention, it is possible to add ink simply and conveniently to a reservoir 5, 105, the added amount of ink corresponding to the needs and not to the capacity of the reservoir.

It is also possible to change the color of the ink simply, which allows the same distribution module 1 to be used for several colors. Moreover, it is possible to use pre-filled cartridges, which avoids handling the ink. It is also possible to prevent the ink from remaining in the open air for a long time, which avoids the risk of contamination by an airborne pathogen.

The shapes proposed for the module 1 are adapted to allow simple and rapid cleaning by dipping all or part of the distributing system in a cleaning liquid. It is therefore very simple to change color.

The device described above is intended to be used for permanent makeup or tattooing. However, other applications are possible using a similar device, for example the injection of a medical product, a cosmetic product, a hair product, or a veterinary product.

Of course, the present invention is not limited to the embodiments described above as examples; it extends to other variants. In particular, it is important to note that the various elements described above can be combined with each other differently from the embodiments that have been shown. For example, a decompression chimney can be combined with any shape of slit(s).

1. A distribution-module module for a device intended for applying a coloring agent for permanent makeup or tattooing, said distribution module comprising:
a channel,
at least one needle, arranged in said channel,
a plunger, arranged in said channel, upstream of said needle, said needle being secured in translation to said plunger, the assembly formed by said needle and said plunger being movable longitudinally in said channel between a retracted position, in which a distal end of the needle is located inside said channel, and an extended position, in which the distal end of the needle protrudes from said channel,
a product reservoir, said reservoir comprising a lumen opening into said channel, and
an automatic valve formed by the cooperation between at least one slit, arranged on the periphery of said plunger, and said lumen, said at least one slit being arranged so as to be located in line with said lumen when said plunger is in a first intermediate product supply zone, located between the retracted position and the extended position, the lumen being closed by said plunger outside said first intermediate product supply zone.

2. The distribution module according to claim 1, comprising a front chamber, arranged in said channel, downstream of said lumen, said slit being able to be located in said front chamber when said plunger is in a second intermediate product supply zone.

3. The distribution module according to claim 1, comprising one or more decompression chimneys opening into said front chamber.

4. The distribution module according to claim 3, wherein said decompression chimney comprises a set of grooves to allow, by capillary action, the product to rise up the decompression chimney.

5. The distribution module according to claim 3, wherein said decompression chimney is integrated into said reservoir.

6. The distribution module according to claim 3, wherein the cooperation of the slit and the lumen further allows air to return to the reservoir.

7. The distribution module according to claim 1, wherein said lumen is shut off by plug before said reservoir is used.

8. The distribution module according to claim 1, wherein said plunger comprises at least one slit having a constant shape extending over the entire circumference of the plunger.

9. The distribution module according to claim 1, wherein said plunger has a cylindrical shape, said plunger comprising several slits of different dimensions, arranged at different positions around the circumference of the plunger, the plunger being rotatable about its axis, to allow selection of the slit to be used for supplying the product.

10. The distribution module according to claim 1, comprising several needles arranged in the same plane, said slit having a substantially rectangular shape and being arranged on said plunger so as to be located in line with said lumen when said plunger is located in said first intermediate product supply zone.

11. The distribution module according to claim 1, wherein the reservoir comprises a bottom, the reservoir and/or said bottom being inclined relative to the axis of said channel.

12. The distribution module according to claim 11, wherein the angle of inclination between the bottom of said reservoir and the axis of said channel is comprised between 5°and 55°.

13. The distribution module according to claim 11, wherein the bottom of the reservoir comprises a set of grooves to allow, by capillary action, the rise of product to the bottom of said reservoir.

14. The distribution module according to claim 1, wherein the reservoir is removable.

15. The distribution module according to claim 14, wherein said removable reservoir comprises a member for connecting said removable reservoir to said distribution module, said connecting member comprising a shutter element for said lumen, movable between a closed position, in which the shutter element shuts off said opening and an open position, in which the shutter element releases said opening the shutter element being displaced from said closed position to said open position when said removable reservoir is mounted on said distribution module.

16. The distribution module according to claim 1, wherein said reservoir comprises means for distributing the product in said reservoir.

17. The distribution module according to claim 1, wherein said reservoir comprises a plug removably mounted on a filling opening of said reservoir.

18. The distribution module according to claim 1, wherein said reservoir comprises cover allowing to close a filling opening of said reservoir.

19. The distribution module according to claim 15, wherein said reservoir comprises an air inlet hole.

20. The distribution module according to claim 19, wherein said shutter element is arranged to close the air inlet hole when it is in said closed position, said shutter element being arranged to release the air inlet hole when it is in said open position.

21. The distribution module according to claim 1, wherein the front part of said channel has the shape of an elongated spout.

22. A device intended for applying a coloring agent for permanent makeup or tattooing, this device being wherein it comprises a distribution module according to claim 1 and a module for actuating the needle of the distribution module.
